(19) United States
(12) Patent Application Publication
(10) Pub. No.: US 2019/0099061 A1
(43) Pub. Date: Apr. 4, 2019

(54) ENDOSCOPE

(71) Applicant: OLYMPUS CORPORATION, Tokyo (JP)

(72) Inventor: Yosuke ISOBE, Tokyo (JP)

(73) Assignee: OLYMPUS CORPORATION, Tokyo (JP)

(21) Appl. No.: 16/207,257

(22) Filed: Dec. 3, 2018

Related U.S. Application Data
(63) Continuation of application No. PCT/JP2017/012772, filed on Mar. 28, 2017.

(30) Foreign Application Priority Data
Jun. 7, 2016 (JP) 2016-113458

(51) Int. Cl. A61B 1/005; A61B 1/06

(52) U.S. Cl. CPC A61B 1/0055; A61B 1/0052; G02B 23/2476; A61B 1/0661; A61B 1/0057

(57) ABSTRACT

An endoscope includes: a bending portion that is provided at a distal end of an insertion section, has plural bending pieces consecutively provided, and is bendable in at least one direction; a flexible tube provided at a proximal end side of the bending portion with flexibility; a control body that is provided at a proximal end side of the flexible tube and receives operation causing the bending portion to be bent; a wire that is inserted through the bending portion and the flexible tube and transmits the operation received by the control body to the distal end of the insertion section; a protruding portion being a plate-like member provided on the wire; and a holding portion that is provided on an inner peripheral surface of the bending piece and holds the protruding portion movably in a direction that the insertion section extends.

### CROSS-REFERENCE TO RELATED APPLICATION

This application is a continuation of PCT International Application No. PCT/JP2017/012772 filed on Mar. 28, 2017 which claims the benefit of priority from Japanese Patent Application No. 2016-113458, filed on Jun. 7, 2016, the entire contents of which are incorporated herein by reference.

### BACKGROUND

The present disclosure relates to an endoscope.

An endoscope, which is inserted into a subject and is for observation of a part to be examined, has been known, and has been widely used in the medical field. There is an endoscope having a bendable bending portion arranged at a distal end of an insertion section to be inserted into a subject (for example, see Japanese Patent Application Laid-open No. 2011-055972). In this endoscope, by operating a control body, a wire that has been inserted through the bending portion is pulled toward a proximal end of the endoscope and the bending portion is bent, the control body being provided consecutively to the proximal end.

### SUMMARY

According to one aspect of the present disclosure, there is provided an endoscope comprising: a bending portion that is provided at a distal end of an insertion section inserted into a subject, the bending portion including bending pieces consecutively provided and being bendable in at least one direction; a flexible tube that is provided at a proximal end side of the bending portion and has flexibility; a control body that is provided at a proximal end side of the flexible tube, the control body being configured to receive operation that causes the bending portion to be bent; a wire that is inserted through the bending portion and the flexible tube, the wire being configured to transmit the operation received by the control body to the distal end of the insertion section; a protruding portion that is a plate-like member provided on the wire; and a holding portion that is provided on an inner peripheral surface of the bending piece, the holding portion being configured to hold the protruding portion movably in a direction that the insertion section extends.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram illustrating a structure of an endoscope according to a first embodiment;

FIG. 2 is a sectional view of a bending portion of the endoscope illustrated in FIG. 1, the sectional view in an insertion direction;

FIG. 3 is a diagram illustrating a wire and protruding portions illustrated in FIG. 2;

FIG. 4 is a sectional view of the bending portion of the endoscope illustrated in FIG. 1, the sectional view being orthogonal to the insertion direction;

FIG. 5 is a sectional view of another design example of the bending portion according to the first embodiment, the sectional view being orthogonal to the insertion direction;

FIG. 6 is an enlarged view of a part of a cross section of a bending portion of an endoscope according to a first modified example of the first embodiment, the cross section being orthogonal to the insertion direction;

FIG. 7 is an enlarged view of a part of a cross section of a bending portion of an endoscope according to a second modified example of the first embodiment, the cross section being orthogonal to the insertion direction;

FIG. 8 is a diagram illustrating a wire and a protruding portion illustrated in FIG. 7;

FIG. 9 is a diagram for explanation of a manufacturing method for wires and protruding portions illustrated in FIG. 8;

FIG. 10 is an enlarged view of a part of a cross section of a bending portion of an endoscope according to a second embodiment, the cross section being orthogonal to the insertion direction;

FIG. 11 is a diagram illustrating a wire and a protruding portion illustrated in FIG. 10; and

FIG. 12 is a diagram illustrating a cross section of a bending portion of a known endoscope.

### DETAILED DESCRIPTION

Hereinafter, by reference to the drawings, embodiments of an endoscope according to the present disclosure will be described. The present disclosure is not limited by these embodiments. The present disclosure is generally applicable to endoscopes, each of which includes a wire that causes a bending portion to be bent. For example, the present disclosure is even applicable to an ultrasonic endoscope including an ultrasound transducer at a distal end of an insertion section thereof.

Further, throughout the drawings, the same reference signs are each assigned to elements that are the same or corresponding to one another, as appropriate. Furthermore, the drawings are schematic, and a relation between dimensions of each element, and ratios among the elements may be different from the actual relation and ratios. These relation and ratios may also differ among the drawings.

### First Embodiment

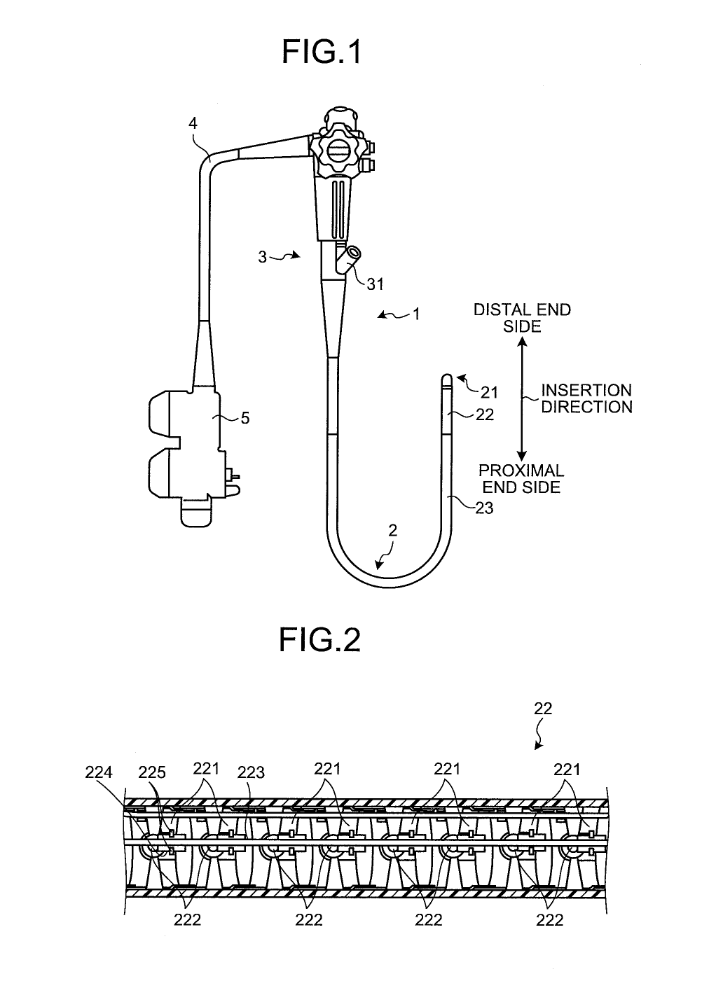

FIG. 1 is a schematic diagram illustrating a structure of an endoscope according to a first embodiment. An endoscope 1 includes: an insertion section 2, which has an imaging unit arranged at a distal end of the insertion section 2, and which is inserted into a subject; a control body 3 that is provided consecutively to a proximal end of the insertion section 2; a universal cord 4 extending out from a side portion of the control body 3; and a connector unit 5, which is provided consecutively to the universal cord 4, and which is connected to an observation apparatus that controls the endoscope 1 and to a light source device for supply of illumination light to the endoscope 1. In this specification, as illustrated in FIG. 1, a direction that the insertion section 2 is inserted will be referred to as an “insertion direction”, and an insertion direction distal end side (the upper side in FIG. 1) will be referred to as a “distal end side”, and an insertion direction proximal end side (the lower side in FIG. 1) will be referred to as a “proximal end side”.

The insertion section 2 has a distal end portion 21 provided at a distal end of the insertion section 2; a bending portion 22 that is provided at a proximal end side of the distal end portion 21 and is bendable; and a flexible tube 23 that is provided at a proximal end side of the bending portion 22 and has flexibility. A proximal end of the flexible tube 23 is provided consecutively to a distal end of the control body 3. The distal end portion 21 has an opening provided therein, through which a surgical tool protrudes inside the subject.

FIG. 2 is a sectional view of the bending portion of the endoscope illustrated in FIG. 1, the sectional view in the insertion direction. The bending portion 22 has plural bending pieces 221 that are consecutively provided, and pins 222 that connect between the bending pieces 221. The bending portion 22 and the flexible tube 23 have a wire 223 inside and inserted therethrough. The wire 223 has a distal end connected to the distal end portion 21 and a proximal end connected to the control body 3, and transmits operation received by the control body 3 to the distal end of the insertion section 2. Specifically, when the wire 223 is pulled to the proximal end side, the bending pieces 221 respectively rotate around the pins 222, and the bending portion 22 is thus bent.

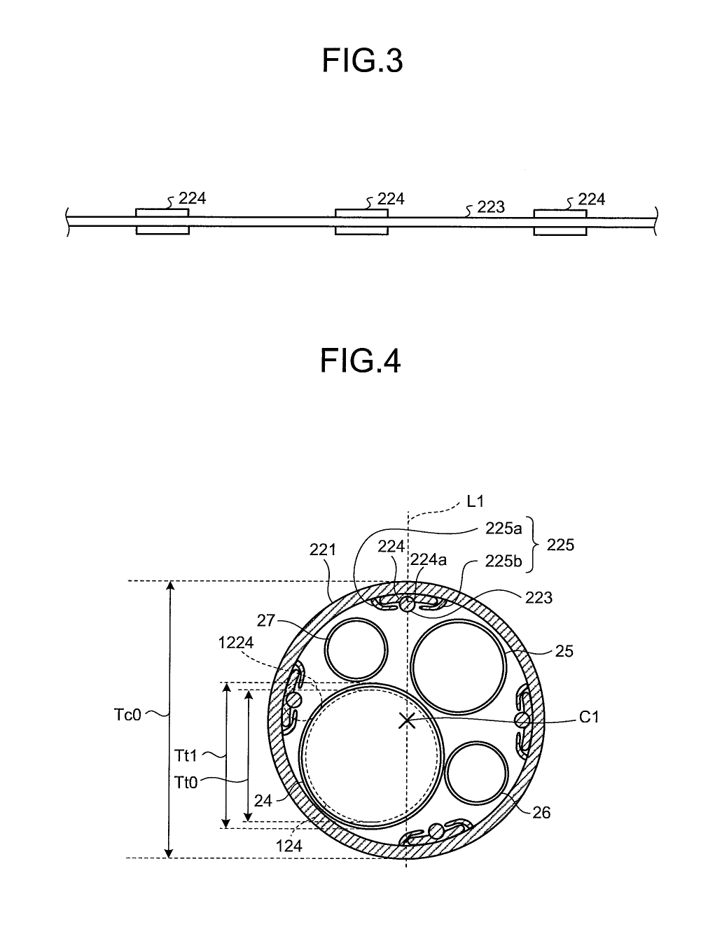

FIG. 3 is a diagram illustrating the wire and protruding portions illustrated in FIG. 2. The wire 223 has plural protruding portions 224 fixed thereto by welding.

FIG. 4 is a sectional view of the bending portion of the endoscope illustrated in FIG. 1, the sectional view being orthogonal to the insertion direction. Each of the protruding portions 224 is a plate-like member that has been bent according to an inner peripheral surface of the bending piece 221. The protruding portion 224 has a concave portion 224a formed therein, and the wire 223 is fixed to this concave portion 224a. As a result, on a plane orthogonal to the insertion direction illustrated in FIG. 4, the protruding portion 224 is in a state of protruding in directions away from a straight line L1 joining the center of the bending piece 221 and the wire 223.

On the inner peripheral surface of the bending piece 221, a holding portion 225 that is bent like hooks is provided. The holding portion 225: has a first holding portion 225a that holds one end of the protruding portion 224, and a second holding portion 225b that holds the other end of the protruding portion 224; and holds the protruding portion 224 movably in the insertion direction. The first holding portion 225a and the second holding portion 225b are arranged apart from each other, with the wire 223 interposed therebetween. Spaces that are continuous in the insertion direction are respectively formed between the first holding portions 225a and the inner peripheral surfaces of the bending pieces 221 and between the second holding portions 225b and the inner peripheral surfaces of the bending pieces 221, and the protruding portions 224 are respectively inserted in these spaces. The wire 223 is arranged between these two spaces, and at least a central portion of the wire 223 is arranged outside these spaces.

Further, arranged inside the bending pieces 221 are: a surgical tool insertion passage 24, through which a surgical tool, such as a puncture needle, is inserted; a signal cable 25 that transmits therethrough image data captured at the distal end of the insertion section 2; a light guide 26 that propagates therethrough the illumination light supplied from the light source device to the distal end of the insertion section 2; and a gas-or-water feeding passage 27 for supply of gas or liquid to the distal end of the insertion section 2.

The control body 3 receives operation that causes the bending portion 22 to be bent. Further, provided in the control body 3 is a surgical tool insertion port 31 that is an entrance of the surgical tool insertion passage 24.

In FIG. 4, on the straight line L1 joining the center C1 of the bending piece 221 and the wire 223, the protruding portion 224 is arranged only on one side of the wire 223. In contrast, in FIG. 12 illustrating a known endoscope, on a straight line L0 joining the center C0 of a bending piece 1221 and a wire 1223, a wire guide 1224 is arranged on both sides of the wire 1223. As a result, according to the first embodiment, as compared with the known endoscope, the amount of protrusion of the structure including the wire 223, the protruding portions 224, and the holding portions 225 to the inside of the bending pieces 221 is small.

Thus, according to the first embodiment, as illustrated in FIG. 4, for example, a thickness Tt1 of the surgical tool insertion passage 24 is able to be made larger than a thickness Tt0 of a known surgical tool insertion passage 124 illustrated with a broken line in FIG. 4. In contrast, in the known endoscope, since the wire guide 1224 and the surgical tool insertion passage 124 interfere with each other, the thickness of the surgical tool insertion passage 124 is unable to be made large. As described above, according to the first embodiment, since a region where the structure to be inserted in the bending portion 22 is arranged is able to be enlarged, this endoscope 1 is an endoscope that is large in freedom of design inside the bending portion 22 and that enables the bending portion 22 to be designed suitably.

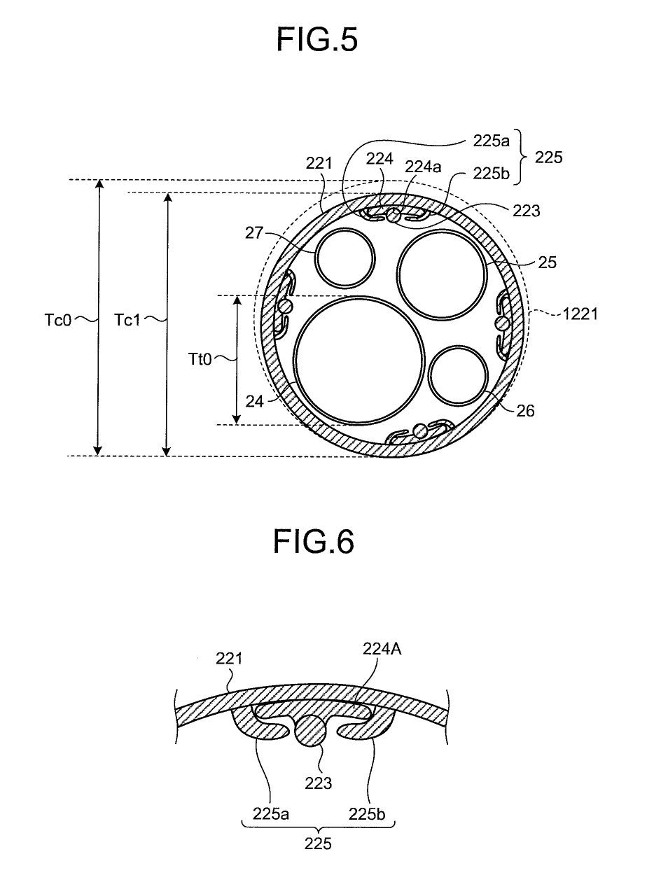

The design of the bending portion 22 is not limited to the structure in FIG. 4. FIG. 5 is a sectional view of another design example of the bending portion according to the first embodiment, the sectional view being orthogonal to the insertion direction. As illustrated in FIG. 5, a thickness Tc1 of the bending portion 22 maybe made smaller than a thickness Tc0 of the known bending piece 1221 illustrated with a broken line in FIG. 5. According to the first embodiment, the diameter of the insertion section 2 including the bending portion 22 is thus able to be reduced, too.

Further, as illustrated in FIG. 3, the structure where the protruding portions 224 having the same length in the insertion direction are arranged at equal intervals has been described above, but the first embodiment is not limited to this structure. For example, in a middle portion of the bending portion 22, the middle portion being where curvature of the bending portion 22 is large upon bending operation, the lengths of the protruding portions 224 in the insertion direction may be made shorter. Furthermore, for example, at the proximal end side of the bending portion 22, the proximal end side being where the amount of movement of the wire 223 to the proximal end side is large upon the bending operation, the lengths of the protruding portions 224 in the insertion direction may be made longer than those at the distal end side.

### FIRST MODIFIED EXAMPLE

FIG. 6 is an enlarged view of a part of a cross section of a bending portion of an endoscope according to a first modified example of the first embodiment, the cross section being orthogonal to the insertion direction. As illustrated in FIG. 6, a protruding portion 224A does not have a concave portion formed therein. As illustrated in FIG. 6, the protruding portion 224A may be configured without a concave portion formed therein. In this structure also, on a straight line joining the center of the bending piece 221 and the wire 223, the protruding portion 224A is arranged only on one side of the wire 223. As a result, according to the first modified example, the amount of protrusion of the structure including the wire 223, the protruding portion 224A, and the holding portion 225 to the inside of the bending piece 221 is small.

### SECOND MODIFIED EXAMPLE

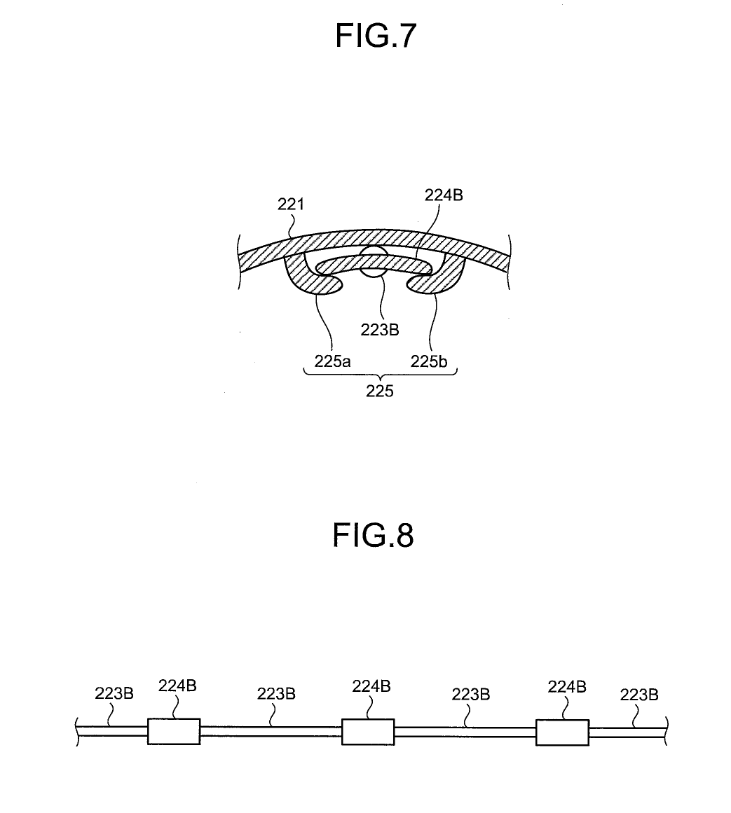

FIG. 7 is an enlarged view of a part of a cross section of a bending portion of an endoscope according to a second modified example of the first embodiment, the cross section being orthogonal to the insertion direction. FIG. 8 is a diagram illustrating a wire and a protruding portion illustrated in FIG. 7. Illustrated in FIG. 7 and FIG. 8 is a structure where plural wires 223B and plural protruding portions 224B are alternately connected to each other in the insertion direction. In this structure, since the thickness of the protruding portions 224B is less than the thickness of the wires 223B on a plane orthogonal to the insertion direction, the amount of the protrusion to the inside of the bending piece 221 is able to be made even smaller.

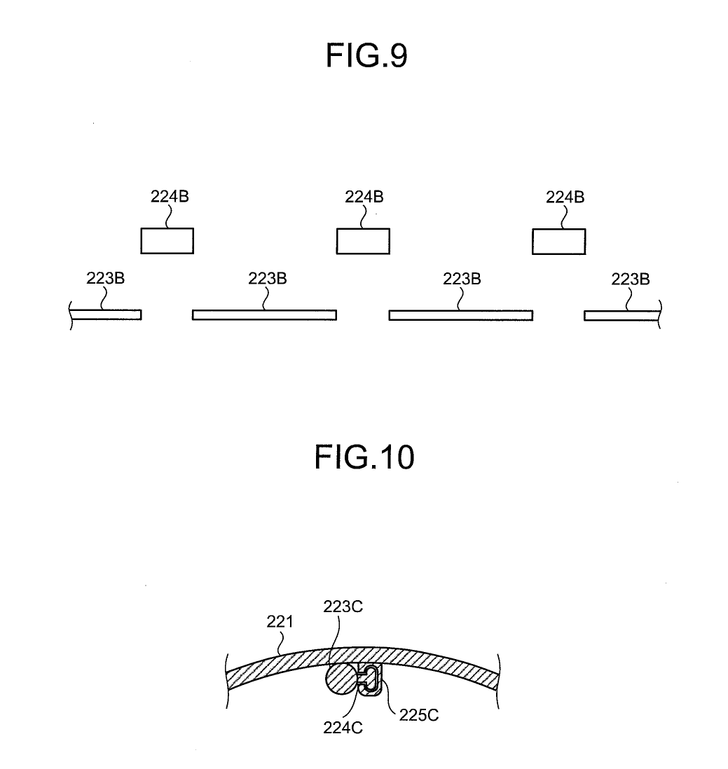

FIG. 9 is a diagram for explanation of a manufacturing method for the wires and protruding portions illustrated in FIG. 8. As illustrated in FIG. 9, the structure in FIG. 8 may be manufactured by: preparation of the short plural wires 223B and the plural protruding portions 224B; and joining of these together by welding.

### Second Embodiment

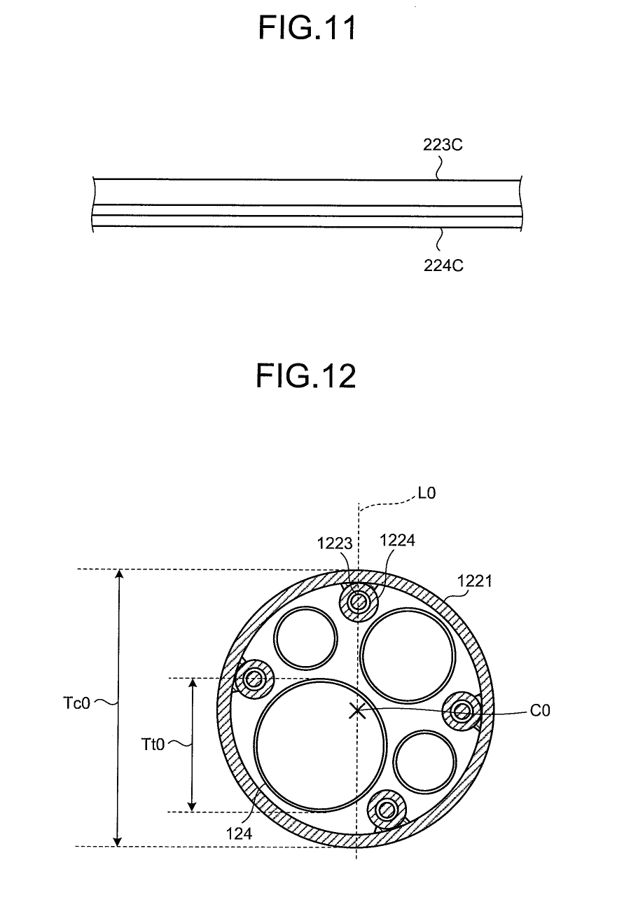

FIG. 10 is an enlarged view of a part of a cross section of a bending portion of an endoscope according to a second embodiment, the cross section being orthogonal to the insertion direction. FIG. 11 is a diagram illustrating a wire and a protruding portion illustrated in FIG. 10. On a plane illustrated in FIG. 10 and orthogonal to the insertion direction, a protruding portion 224C protrudes in one of directions away from a straight line joining the center of the bending piece 221 and a wire 223C. A distal end of the protruding portion 224C is T-shaped. Further, the protruding portion 224C has, as illustrated in FIG. 11, a shape that is continuous in the insertion direction.

A holding portion 225C has a shape that fits with the T-shaped protruding portion 224C. The structure other than the wire 223C, the protruding portion 224C, and the holding portion 225C may be the same as that of the first embodiment, and thus description thereof will be omitted. As described above, the protruding portion 224C may be provided only on the one side. In this structure, the protruding portion 224C is not arranged on both sides of the wire 223C on the straight line joining the center of the bending piece 221 and the wire 223C. As a result, according to the second embodiment, the amount of protrusion of the structure including the wire 223C, the protruding portion 224C, and the holding portion 225C to the inside of the bending piece 221 is even smaller.

With respect to the embodiments, the structure including four wires has been described, but the embodiments are not limited to this structure. For example, the embodiments are applicable to a structure including one or two wires and including a bending portion that is bendable in one direction or two directions.

Further, with respect to the embodiments, the structure where the wire is fixed to the protruding portion by welding has been described, but the embodiments are not limited to this structure. For example, a structure corresponding to a wire and protruding portions may be integrally formed, such that a cross sectional shape that is the same as that of FIG. 4, FIG. 6, or FIG. 10 is obtained.

According to the present disclosure, an endoscope enabling its bending portion to be designed suitably is able to be realized.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An endoscope, comprising:
a bending portion that is provided at a distal end of an insertion section inserted into a subject, the bending portion including bending pieces consecutively provided and being bendable in at least one direction;
a flexible tube that is provided at a proximal end side of the bending portion and has flexibility;
a control body that is provided at a proximal end side of the flexible tube, the control body being configured to receive operation that causes the bending portion to be bent;
a wire that is inserted through the bending portion and the flexible tube, the wire being configured to transmit the operation received by the control body to the distal end of the insertion section;
a protruding portion that is a plate-like member provided on the wire; and
a holding portion that is provided on an inner peripheral surface of the bending piece, the holding portion being configured to hold the protruding portion movably in a direction that the insertion section extends.

2. The endoscope according to claim 1, wherein the wire is arranged outside a space formed by the inner peripheral surface of the bending piece and the holding portion.

3. The endoscope according to claim 1, wherein
the protruding portion protrudes in both of directions away from a straight line joining a center of the bending piece and the wire on a plane orthogonal to a direction that the insertion section extends; and
the holding portion includes:
a first holding portion configured to hold one end of the protruding portion; and
a second holding portion configured to hold another end of the protruding portion, the second holding portion being arranged apart from the first holding portion.

4. The endoscope according to claim 1, wherein a thickness of the protruding portion is smaller than a thickness of the wire on a plane orthogonal to a direction that the insertion section extends.

5. The endoscope according to claim 1, wherein the protruding portion is formed continuously in a direction that the insertion section extends.

6. The endoscope according to claim 1, wherein the protruding portion includes a concave portion formed therein, and the wire is fixed to the concave portion.

7. The endoscope according to claim 1, further comprising one or more protruding portions besides said protruding portion, wherein the protruding portions are arranged on the wire.

8. The endoscope according to claim 7, further comprising one or more wires besides said wire, wherein the wires and the protruding portions are connected to each other.
